(19) United States
(12) Patent Application Publication
(10) Pub. No.: US 2014/0180168 A1
(43) Pub. Date: Jun. 26, 2014

(54) GUIDEWIRE WITH TOUCH SENSOR

(71) Applicant: VOLCANO CORPORATION, San Diego, CA (US)

(72) Inventor: Bret Millett, Folsom, CA (US)

(73) Assignee: VOLCANO CORPORATION, San Diego, CA (US)

(21) Appl. No.: 14/136,151

(22) Filed: Dec. 20, 2013

Related U.S. Application Data
(60) Provisional application No. 61/745,307, filed on Dec. 21, 2012.

(51) Int. Cl. A61B 5/107; A61B 5/00

(52) U.S. Cl. CPC A61B 5/1076; A61B 5/6852; A61B 5/6851; A61B 5/1079
USPC 600/587

(57) ABSTRACT

The present invention generally relates to methods and devices for sensing the surface of a luminal wall. The invention can involve an elongated body configured to fit within the lumen of a vessel and at least one touch sensor located on the elongated body.

### RELATED APPLICATIONS

This application claims priority to U.S. Provisional Application No. 61/745,307, filed Dec. 21, 2012, which is incorporated by reference herein in its entirety.

### FIELD OF THE INVENTION

The present invention generally relates to devices and methods for sensing surfaces of luminal walls.

### BACKGROUND

Cardiovascular disease frequently arises from the accumulation of atheromatous deposits on inner walls of vascular lumen, particularly the arterial lumen of the coronary and other vasculature, resulting in a condition known as atherosclerosis. These deposits can have widely varying properties, with some deposits being relatively soft and others being fibrous and/or calcified. In the latter case, the deposits are frequently referred to as plaque. These deposits restrict blood flow, which in severe cases can lead to myocardial infarction.

The assessment and treatment of cardiovascular disease often involves cardiac catheterization. In this medical procedure, a catheter is inserted into a chamber or blood vessel of the heart in order to diagnose or treat certain conditions. For example, cardiac catheterization may be used to evaluate areas in which plaque is constricting arterial vessels. Subsequent treatment may include angioplasty, in which a small balloon is put through the catheter into the blocked vessel and used to compress plaque upon inflation. Other types of treatment can include the delivery and placement of a stent into the vessel via the catheter.

A thin, flexible wire known as a guide wire is typically used to lead the catheter to the appropriate location. Prior to insertion of the catheter, the guide wire is inserted into a blood vessel and advanced towards the heart. The catheter slides over the guide wire into the coronary vessels to the desired location.

When advancing the catheter or guide wire, it is critical not to over-advance the device or to use excessive force when advancing the device, which can lead to damaging delicate blood vessels or tissue in the surrounding vasculature. Unfortunately, conventional devices have not yet addressed this need for optimal sensitivity when conducting such procedures.

### SUMMARY

The present invention generally relates to devices and methods for sensing surfaces of luminal walls. More specifically, the invention relates to devices that include an elongated body configured for insertion into a lumen of a vessel equipped with a touch sensor. The touch sensor provides feedback to the operator when the device is advanced through the vessel. When the touch sensor comes into contact with an object or a surface, feedback is transmitted to the operator, who can then determine whether to advance the device further or to proceed using less force. In this manner, damage that may occur to blood vessels or surrounding tissue during cardiac catheterization is avoided. In addition, the present invention provides the operator with greater sensitivity during these delicate procedures.

Although the touch sensor can operate through any means known in the art, in certain aspects of the invention, the touch sensor operates through the use of optical fibers. In further aspects of the invention, the optical fiber includes a fiber Bragg grating (FBG). When, for example, the tip of the optical fiber comes into contact with an object, the fiber is slightly deformed and the FBGs inside the fiber is stretched or compressed. By monitoring the resulting change in the wavelength of light passing through the FGBs, the magnitude and position of the applied force can be determined. In certain aspects of the invention, the optical fiber includes a blazed FBG, in which the impressed index changes of the FBG on the optical fiber are arranged at non-perpendicular angles relative to the longitudinal axis of the optical fiber.

Any device configured for insertion into a vessel lumen is useful for practicing the invention, however, in certain aspects, the invention encompasses a guide wire or catheter configured with a touch sensor. In other aspects, both the guide wire and catheter are equipped with touch sensors. In either of these embodiments, the touch sensor affords the operator greater precision and sensitivity in placing the device. For example, in using a guide wire equipped with touch sensor, the operator can place the guide wire appropriately without damaging the surrounding tissue. The operator can then safely proceed with advancing the catheter.

Devices in accordance with the invention may include single or multiple sensors which can be positioned anywhere along the device. For example, the device may include a single touch sensor positioned at a distal end of the guide wire, such as the tip. In other aspects, the device may include numerous touch sensors positioned at the sides of the guide wire. In such embodiments, the operator can avoid colliding the length of the guide wire against the vessel wall. The exact number and positioning of the sensors can be adjusted as desired.

Elongated devices having touch sensors of the invention may be incorporated into systems for the treatment of patients. Such systems may include a detection module configured to receive a signal from the touch sensor. Such detection modules are known in the art and include for example, FBG interrogators. In certain aspects of the invention, the detection module receives a signal produced from a distortion of the FBGs and converts that signal into useful feedback for the operator, so that the operator knows to cease advancing the guide wire along that route. Feedback can be provided in many forms that can be used alone or in combination with one another. For example, feedback may include audible feedback, such as a beeping or buzzing sound that is emitted when the sensor contacts an object. Feedback may also be visual, such as a light that blinks or flashes when contact occurs. Feedback can also include tactile feedback, such as a grip held by the operator that vibrates whenever contact occurs.

### BRIEF DESCRIPTION OF THE DRAWINGS

FIG. 1 depicts an optical fiber suitable for use with the provided touch sensing devices.

FIG. 2 depicts an embodiment of a sensing element that includes a piezoelectric element.

FIGS. 3 and 4 depict a sensing element that uses Fiber Bragg Gratings to generate acoustic energy.

FIG. 5 is a block diagram generally illustrating an image assembly of the invention and several associated interface components.

FIG. 6 is a block diagram illustrating another example of an imaging assembly of the invention and associated interface components.

FIG. 7 shows a cross-section of an exemplary sensing guide wire, including a plurality of imaging elements.

FIG. 8 depicts a distal portion of an exemplary sensing guide wire.

FIG. 9 illustrates a cross-sectional view of an exemplary touch sensing catheter.

FIG. 10 depicts another exemplary embodiment of an imaging catheter.

FIG. 11 depicts a close-up perspective of an exemplary touch sensor.

### DETAILED DESCRIPTION

The invention generally relates to devices and methods for sensing the surface of a luminal wall. More specifically, the invention relates to devices that include an elongated body configured for insertion into a lumen of a vessel and a touch sensor located on the elongated body. The touch sensor provides feedback to the operator as the device is advanced through the vessel. When the touch sensor comes into contact with an object or a surface, feedback is transmitted to the operator, who can then determine whether to stop advancing the device or to proceed using less force.

Devices and methods of the invention are useful in many applications that require a high degree of sensitivity to measure contact force, such as medical applications. One particular medical application is in cardiac catheterization where it is important for physicians to know the contact force between the catheters and blood vessel walls in order to avoid damaging the delicate blood vessel networks of the patient during an interventional procedure. In the present disclosure, reference is made primarily to touch sensing guide wires, however, it is to be understood that the present invention is equally applicable to other elongated bodies, such as catheters.

The provided touch sensing guide wire can be introduced into a lumen of the body to obtain tactile information regarding the vessel prior to introduction of a catheter. The body lumens generally are diseased body lumens and, in particular, lumens of the vasculature. The tactile information obtained may be used to avoid damaging a region or location of interest within a body lumen. Regions of interest often include some type of defect. The defect, for example, can be a de novo lesion or an in-stent restenosis lesion for example. The devices and methods, however, are also suitable for assessing stenosis of body lumens and other hyperplastic and neoplastic conditions in other body lumens, such as the ureter, the biliary duct, respiratory passages, the pancreatic duct, the lymphatic duct, and the like. In addition, the region of interest can include, for example, a location for stent placement or a location including plaque or diseased tissue that needs to be removed.

Once the touch sensing guide wire is in place, a catheter, such as an imaging catheter, can be introduced over the guide wire to the location of interest. The imaging catheter can obtain images of the intraluminal surface as the imaging catheter moves towards the region of interest, which allows the imaging catheter to be precisely placed into the region of interest and provides for tracking of the imaging catheter along the path of the guide wire. In addition, the imaging catheter can be used to obtain different imaging views of the region of interest. Imaging catheters are known in the art, and include for example, the EAGLE EYE phased array IVUS catheter and the REVOLUTION pull-back IVUS catheter, both of which are produced by Volcano Corporation (San Diego, Calif.).

In certain aspects, the catheter may also serve as a delivery catheter for delivery of some type of a therapeutic device, such as a stent, ablator, balloon, and the like. During the procedure, the touch sensing guide wire may be used to identify the appropriate location and the delivery catheter used to deliver the device to the appropriate location.

The touch sensing guide wire is configured for intraluminal introduction into a target body lumen. The dimensions and other physical characteristics of the guide wire will vary significantly depending on the body lumen that is to be accessed. In addition, the dimensions can depend on the placement and number of sensing elements included on the touch sensing guide wire.

For the touch sensing guide wire, the sensing element can be formed as, or integrated into, the body of the imaging guide wire, circumscribe the guide wire, and/or run along the body of the guide wire. The touch sensing guide wire may also include an outer support structure or coating surrounding the imaging elements. The touch sensing guide wire including the sensing element (for example, an optical fiber and transducer material) and, in certain embodiments, the surrounding support structure can have a total outside diameter of less than 1 mm, preferably less than 300 micron (less than about 1 French).

The provided touch sensing guide wire bodies may include a solid metal or polymer core. Suitable polymers include polyvinylchloride, polyurethanes, polyesters, polytetrafluoroethylenes (PTFE), silicone rubbers, natural rubbers, and the like. Preferably, at least a portion of the metal or polymer core and other elements that form the imaging guide wire body are flexible.

In certain embodiments, a touch-sensing catheter is provided. The sensing element can form or be integrated within the body of the catheter, circumscribe the catheter, placed on a distal end face of the catheter, and/or run along the body of the catheter. The sensing catheter may also include an outer support structure or coating surrounding the imaging elements. Sensing catheter bodies intended for intravascular introduction will typically have a length in the range from 50 cm to 200 cm and an outer diameter in the range from 1 French to 12 French (0.33 mm: 1 French), usually from 3 French to 9 French. In the case of coronary catheters, the length is typically in the range from 125 cm to 200 cm, the diameter is preferably below 8 French, more preferably below 7 French, and most preferably in the range from 2 French to 7 French.

Catheter bodies will typically be composed of an organic polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase rotational strength, column strength, toughness, pushability, and the like. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques. Preferably, at least a portion of the catheter body is flexible.

The touch sensing guide wire or catheter of the invention may include a detection module or imaging assembly. Any imaging assembly may be used with devices and methods of the invention, such as optical-acoustic imaging apparatus, intravascular ultrasound (IVUS) or optical coherence tomography (OCT). The imaging assembly is used to send and receive signals to and from the imaging surfaces that form the imaging data.

In preferred embodiments, the imaging assembly is configured to send and receive optical/light signal through touch-sensing element(s) of the device. In certain embodiments of the invention, the touch-sensing element(s) comprise an optical fiber through which the imaging assembly sends and receives optical signals. In certain embodiments, the optical fiber includes a Fiber Bragg Grating within the optical fiber. Further detail regarding the touch sensing elements is provided throughout the present disclosure.

Fiber Bragg Gratings (FBGs) provide a means for measuring the interference between two paths taken by a coherent beam of light. A partially reflecting Fiber Bragg Grating is used to split the incident beam of light into two parts, in which one part of the beam travels along a path that is kept constant (constant path) and another part travels a path for detecting a change (change path). The paths are then combined to detect any interference in the beams. If the paths are identical, then the two paths combine to form the original beam. If the paths are different, then the two parts will add or subtract from each other and form an interference pattern. The Fiber Bragg Grating elements are thus able to sense a change in path length between the constant path and the change path based on received ultrasound or acoustic energy. The detected optical signal interferences can be used to generate an image using any conventional means. In the provided touch sensing devices, when the tip of the optical fiber comes into contact with an object, the fiber is slightly deformed and the Fiber Bragg Gratings inside the fiber is stretched or compressed. By monitoring the resulting change in the wavelength of light passing through the Fiber Bragg Gratings, the magnitude and position of the applied force can be determined.

In certain embodiments, the sensing element includes a piezoelectric element to generate acoustic or ultrasound energy, which provides another means for sensing an object surface and provide enhanced tactile capabilities. In such aspect, the optical fiber of the sensing element may be coated by the piezoelectric element. The piezoelectric element may include any suitable piezoelectric or piezoceramic material. In one embodiment, the piezoelectric element is a poled polyvinylidene fluoride or polyvinylidene difluoride material. The piezoelectric element can be connected to one or more electrodes that are connected to a signal generator that transmits pulses of electricity to the electrodes. The electric pulses cause mechanical oscillations in the piezoelectric element, which generates an acoustic signal. Thus, the piezoelectric element is an electric-to-acoustic transducer. Primary and reflected pulses (i.e. reflected from the sensing medium) are received by the Bragg Grating element and transmitted to an electronic instrument to generate tactile information.

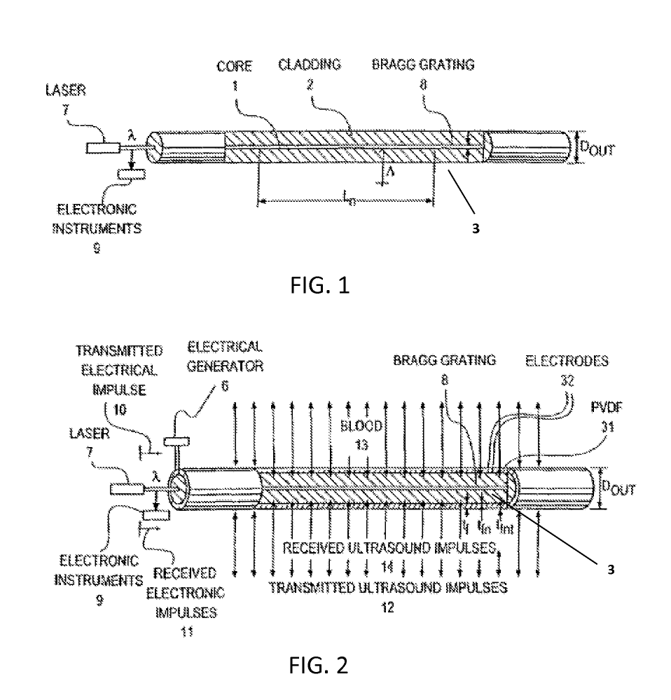

FIG. 2 depicts an embodiment of a touch-sensing element that includes a piezoelectric element. The imaging element includes an optical fiber 3 (such as the optical fiber in FIG. 1) with Fiber Bragg Grating 8 and a piezoelectric element 31. As shown in FIG. 2, an electrical generator 6 stimulates the piezoelectric element 31 (electrical-to-acoustic transducer) to transmit ultrasound impulses 10 to both the Fiber Bragg Grating 8 and the outer medium 13 in which the device is located. For example, the outer medium may include blood when imaging a vessel. Primary and reflected impulses 11 are received by the Fiber Bragg Grating 8 (acting as an acoustic-to-optical transducer). The mechanical impulses deform the Bragg Grating and cause the Fiber Bragg Grating to modulate the light reflected within the optical fiber, which generates an interference signal. The interference signal is recorded by electronic detection instrument 9, using conventional methods. The electronic instrument may include a photodetector and an oscilloscope. Tactile information regarding the contact between the touch sensing device and the object can be generated from these recorded signals. The electronic instruments 9 modulate of light reflected backwards from the optical fiber due to mechanical deformations. The optical fiber with a Bragg Grating described herein and shown in FIG. 1, the imaging element described herein and shown in FIG. 2, and other varying embodiments are described in more detail in U.S. Pat. Nos. 6,659,957 and 7,527,594 and in U.S. Patent Publication No. 2008/0119739, incorporated herein by reference in their entireties.

In another aspect, the imaging element does not require an electrical-to-acoustic transducer to generate acoustic/ultrasound signals. Instead, the imaging element utilizes the one or more Fiber Bragg Grating elements of the optical fiber in combination with an optical-to-acoustic transducer material to generate acoustic energy from optical energy. In this aspect, the acoustic-to-optical transducer (signal receiver) also acts as an optical-to-acoustic transducer (signal generator).

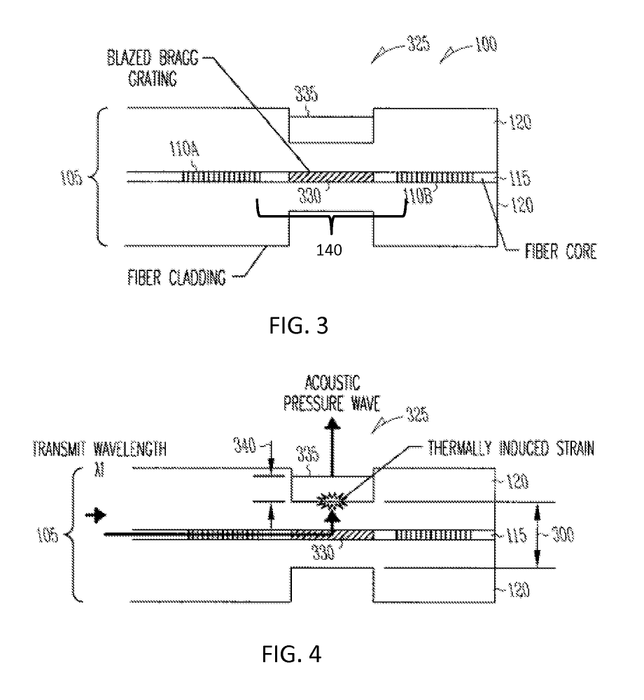

To generate the acoustic energy, the imaging element may include a combination of blazed and unblazed Fiber Bragg Gratings. Unblazed Bragg Gratings typically include impressed index changes that are substantially perpendicular to the longitudinal axis of the fiber core of the optical fiber. Unblazed Bragg Gratings reflect optical energy of a specific wavelength along the longitudinal of the optical fiber. Blazed Bragg Gratings typically include obliquely impressed index changes that are at a non-perpendicular angle to the longitudinal axis of the optical fiber. Blazed Bragg Gratings reflect optical energy away from the longitudinal axis of the optical fiber. FIGS. 3 and 4 depict an imaging element according to this embodiment.

FIG. 3 shows an example of touch sensing element that uses Fiber Bragg Gratings to generate acoustic energy for enhanced tactile capabilities. As depicted in FIG. 4, the touch sensing element 100 includes an optical fiber 105 with unblazed Fiber Bragg Grating 110A and 110B and blazed Fiber Bragg Grating 330 and a photoacoustic material 335 (optical-to-acoustic transducer). The region between the unblazed Fiber Bragg Grating 110A and 110B is known as the strain sensing region 140. The strain sensing region may be, for example, 1 mm in length. The Blazed Fiber Bragg Grating 330 is implemented in the strain sensing region 140. The photoacoustic material 335 is positioned to receive the reflected optical energy from the blazed Fiber Bragg Grating 330. Although not shown, the proximal end of the optical fiber 105 is operably coupled to a laser and one or more electronic detection elements.

In operation and as depicted in FIG. 4, the blazed Fiber Bragg Grating 330 receives optical energy of a specific wavelength λ (1) from a coherent light source, e.g. a laser, and blazed Grating 330 directs that optical energy towards photoacoustic material 335. The received optical energy in the photoacoustic material 335 is converted into heat, which causes the material 335 to expand. Pulses of optical energy sent to the photoacoustic material 335 cause the photoacoustic material 335 to oscillate. The photoacoustic material 335 oscillates, due to the received optical energy, at a pace sufficient to generate an acoustic or ultrasound wave. The acoustic wave is transmitted out to and reflected from the object surface back to the touch sensing element, particularly when the device contacts an object. The acoustic wave reflected from the object surface impinges on photoacoustic transducer 335, which causes a vibration or deformation of photoacoustic transducer 335. This results in a change in length of light path within the strain sensing region 140. Light received by blazed fiber Bragg grating from photoacoustic transducer 135 and into fiber core 115 combines with light that is reflected by either fiber Bragg grating 110A or 110B (either or both may be including in various embodiments). The light from photoacoustic transducer 135 will interfere with light reflected by either fiber Bragg grating 110A or 110B and the light returning to the control unit, and the combined light will exhibit an interference pattern. This interference pattern encodes the tactile information captured by touch sensing element 100. The light 137 can be received into photodiodes within a control unit and the interference pattern thus converted into an analog electric signal. This signal can then be digitized using known digital acquisition technologies and processed, stored, or displayed as tactile information regarding the target treatment site.

Acoustic energy of a specific frequency may be generated by optically irradiating the photoacoustic material 335 at a pulse rate equal to the desired acoustic frequency. The photoacoustic material 335 can be any suitable material for converting optical energy to acoustic energy and any suitable thickness to achieve a desired frequency. The photoacoustic material 335 may have a coating or be of a material that receives acoustic energy over a band of frequencies to improve the generation of acoustic energy by the photoacoustic material and reception of the acoustic energy by the optical fiber sensing region.

In one example, the photoacoustic material 335 has a thickness 340 (in the direction in which optical energy is received from blazed Bragg grating 330) that is selected to increase the efficiency of emission of acoustic energy. In one example, thickness 340 is selected to be about ¼ the acoustic wavelength of the material at the desired acoustic transmission/reception frequency. This improves the generation of acoustic energy by the photoacoustic material.

In a further example, the photoacoustic material is of a thickness 300 that is about ¼ the acoustic wavelength of the material at the desired acoustic transmission/reception frequency, and the corresponding glass-based optical fiber sensing region resonant thickness 300 is about ½ the acoustic wavelength of that material at the desired acoustic transmission/reception frequency. This further improves the generation of acoustic energy by the photoacoustic material and reception of the acoustic energy by the optical fiber sensing region. A suitable photoacoustic material is pigmented polydimethylsiloxane (PDMS), such as a mixture of PDMS, carbon black, and toluene.

The sensing element described and depicted in FIGS. 3 and 4 and other varying embodiments are described in more detail in U.S. Pat. Nos. 7,245.789, 7447,388, 7,660,492, and 8,059,923 and in U.S. Patent Publication Nos. 2010/0087732 and 2012/0108943, all of which are incorporated herein by reference in their entireties.

In certain embodiments, an optical fiber of a sensing element (such as one shown in FIGS. 2-4) can include a plurality of Fiber Bragg Gratings, each with its own unique period (e.g., 0.5 μ), that interact with at least one other transducer. Because each Fiber Bragg Grating can be directed to transmit and receive signals of specific wavelengths, the plurality of Fiber Bragg Gratings in combination with a tunable filter can be used to generate an array of distributed sonars.

Additional components may be used in conjunction with the sensing guide wire or catheter to allow an operator to detect a luminal surface. These additional components are referred to generally as an imaging or detection assembly or module.

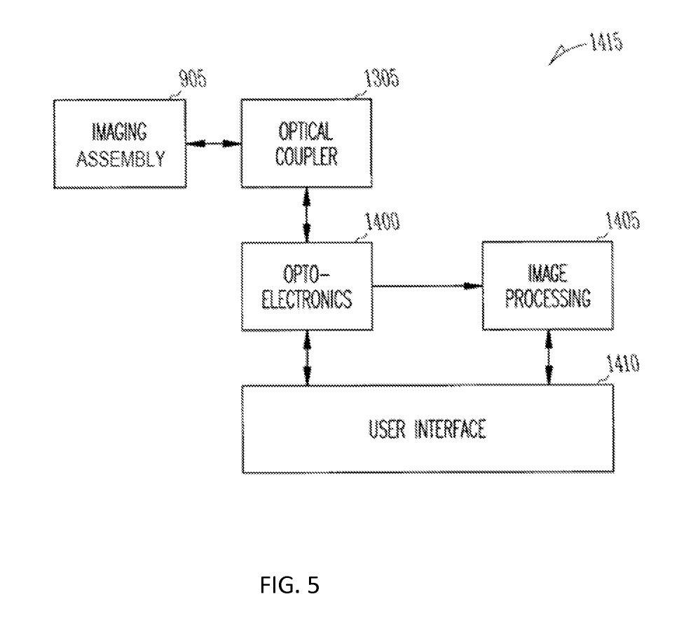

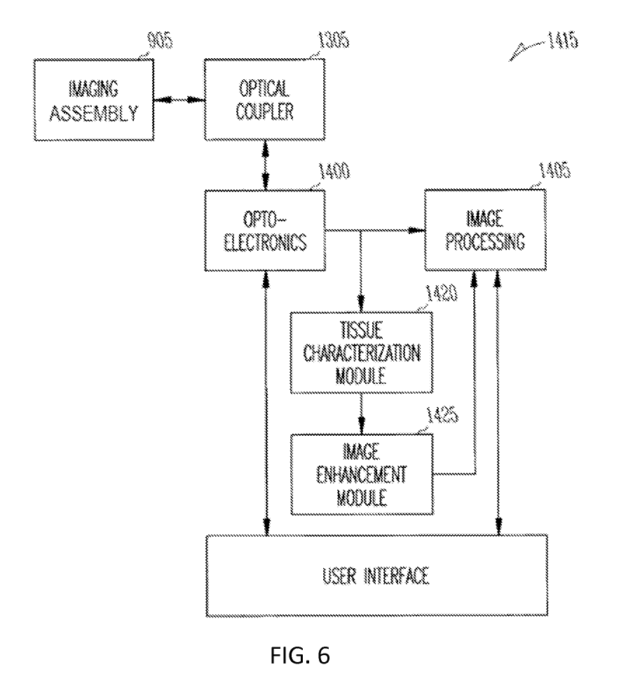

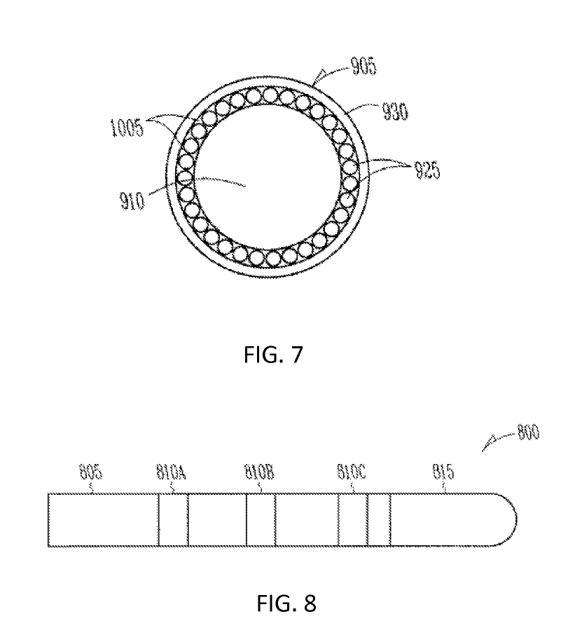

FIG. 5 is a block diagram illustrating generally a detection assembly 905 and several associated interface components. The block diagram of FIG. 6 includes the detection assembly 905 that is coupled by optical coupler 1305 to an optoelectronics module 1400. The optoelectronics module 1400 is coupled to an image processing module 1405 and a user interface 1410 that provides feedback to an operator based on signals received from the touch sensor. Feedback can include an audible signal, such as a beeping sound that is heard when the device contacts a surface. Feedback can also include visual feedback, such as a blinking light that is activated when the device contacts an object. Feedback may also include tactile feedback. For example, the guide wire grip may vibrate in the operator's hand when the device contacts an object. In one example, the system 1415 illustrated in the block diagram of FIG. 5 uses an image processing module 1405 and a user interface 1410 that are substantially similar to existing acoustic imaging systems.

FIG. 6 is a block diagram illustrating generally another example of the detection assembly 905 and associated interface components. In this example, the associated interface components include a tissue (and plaque) characterization module 1420 and an image enhancement module 1425. The invention recognizes that different tissues or areas of tissue as well as various stages of plaque will “feel” different to the touch sensor and therefore, will be able to be distinguished by the provided device on touch alone. In this example, an input of tissue characterization module 1420 is coupled to an output from optoelectronics module 1400. An output of tissue characterization module 1420 is coupled to at least one of user interface 1410 or an input of image enhancement module 1425. An output of image enhancement module 1425 is coupled to user interface 1410, such as the image processing module 1405. In some embodiments signal processing can be used to distinguish different tissues based upon the response of one or more touch sensors. Such signal processing may rely upon a look-up table or precalibration of the touch sensors based upon using similar devices on, for example, cadavers.

In this example, tissue characterization module 1420 processes a signal output from optoelectronics module 1400. In one example, such signal processing assists in distinguishing plaque from nearby vascular tissue. Such plaque can be conceptualized as including, among other things, cholesterol, thrombus, and loose connective tissue that build up within a blood vessel wall. Calcified plaque typically reflects ultrasound better than the nearby vascular tissue, which results in high amplitude echoes. Soft plaques, on the other hand, produce weaker and more texturally homogeneous echoes. Accordingly, when the device contacts such surfaces, the device will provide different feedback to the operator. These and other differences distinguishing between plaque deposits and nearby vascular tissue are detected using tissue characterization signal processing techniques.

For example, such tissue characterization signal processing may include performing a spectral analysis that examines the energy of the returned ultrasound signal at various frequencies. A plaque deposit will typically have a different spectral signature than nearby vascular tissue without such plaque, allowing discrimination there between. Such signal processing may additionally or alternatively include statistical processing (e.g., averaging, filtering, or the like) of the returned ultrasound signal in the time domain. Other signal processing techniques known in the art of tissue characterization may also be applied. In one example, the spatial distribution of the processed returned ultrasound signal is provided to image enhancement module 1425, which provides resulting image enhancement information to image processing module 1405. In this manner, image enhancement module 1425 provides information to user interface 1410 that results in a displaying plaque deposits in a visually different manner (e.g., by assigning plaque deposits a discernible color on the image) than other portions of the image. Other image enhancement techniques known in the art of imaging may also be applied. In a further example, similar techniques are used for discriminating between vulnerable plaque and other plaque, and enhancing the displayed image provides a visual indicator assisting the user in discriminating between vulnerable and other plaque.

A system of the invention may additionally include an opto-electronics module 1400. The opto-electronics module 1400 may include one or more lasers and fiber optic elements. In one example, such as where different transmit and receive wavelengths are used, a first laser is used for providing light to the imaging assembly 905 for the transmitted ultrasound, and a separate second laser is used for providing light to the imaging assembly 905 for being modulated by the received ultrasound. In this example, a fiber optic multiplexer couples each channel (associated with a particular one of the optical fibers 925) to transmit and receive lasers and associated optics. This reduces system complexity and costs.

In one example, the sharing of transmission and reception components by multiple guide wire channels is possible at least in part because the acoustic image is acquired over a relatively short distance (e.g., millimeters). The speed of ultrasound in a human or animal body is slow enough to allow for a large number of transmit/receive cycles to be performed during the time period of one image frame. For example, at an image depth (range) of about 2 cm, it will take ultrasonic energy approximately 26 microseconds to travel from the sensor to the range limit, and back. In one such example, therefore, an about 30 microseconds transmit/receive (T/R) cycle is used. In the approximately 30 milliseconds allotted to a single image frame, up to 1,000 T/R cycles can be carried out. In one example, such a large number of T/R cycles per frame allows the system to operate as a phased array even though each sensor is accessed in sequence. Such sequential access of the photoacoustic sensors in the guide wire permits (but does not require) the use of one set of T/R opto-electronics in conjunction with a sequentially operated optical multiplexer.

In certain aspects, one or more touch sensing elements are incorporated into a touch sensing guide wire. The touch sensing guide wire of the invention allows one to sense through touch a luminal surface prior to introducing a catheter into a body lumen, e.g., a blood vessel. Because the touch sensing guide wire obtains tactile information regarding a luminal surface, an operator can use the provided guide wire to avoid damaging a region of interest within the vasculature by excessive force or over-advancing the guide wire prior to introducing a catheter device. The provided guide wire can also be used to distinguish between different surface types within the vasculature. The one or more sensing elements can be formed around an inner guide wire body, integrated into an inner guide wire body, or form the guide wire body itself. For example, the sensing elements may be positioned along the sides of the guide wire. In other aspects, the sensing element is located at a distal region of the guide wire. The distal region can include the most distal region of the guide wire, for example, the tip. The touch sensing guide wire may include a support structure covering at least a portion of the sensing element. The support structure can include one or more windows that allow the sensing element to send and receive signals that form the tactile data.

In one example, a plurality of sensing elements surrounds an inner guide wire body. FIG. 8 shows a cross-section of the touch sensing guide wire 905 showing a plurality of imaging elements surrounding the inner guide wire body 910. The imaging elements 925 are placed next to each other, parallel to, and along the length of the inner guide wire body 910. The guide wire body 910 can be any suitable flexible material. A binder material 1005 can provide structure support to the sensing elements 925. The sensing elements 925 are optionally overlaid with a protective outer coating 930 that provides for transmission of imaging signals.

Typically, the sensing elements are placed parallel to and along the length of the guide wire. In such aspect, the imaging elements image surfaces substantially perpendicular to the longitudinal axis of the imaging guide wire. However, other configurations may be used. For example, one or more imaging elements may be wrapped around the inner guide wire body. In addition, it is also contemplated at least a portion of the imaging elements are positioned substantially across the longitudinal axis of the guide wire. For example, the imaging elements can be positioned across a distal tip of the imaging guide wire such that the imaging elements image objects or surfaces in front of the imaging guide wire. This position of the imaging elements is described in more detail in co-owned and co-pending application entitled “Chronic Total Occlusion Catheter,” filed as U.S. Provisional Patent Application No. 61/745,358 on Dec. 21, 2012.

In certain embodiments, the imaging guide wire further includes a support structure surrounding the one or more sensing elements. The support structure may include a plurality of windows to allow transmission and reception of signals (e.g. acoustic signals). FIG. 8 depicts a distal portion 800 of an imaging guide wire 805 according to one embodiment. The touch sensing guide wire 805 includes one or more windows 810A, 810B, . . . , 810N. Each window 810 may expose at least a portion of one or more sensing elements. The exposed portion of each sensing element may include one or more acoustic-to-optical transducers (e.g. Fiber Bragg Grating in an optical fiber) that correspond to one or more optical-to-acoustic transducers (i.e. photoacoustic material) or one or more electrical-to-acoustic transducers (i.e. piezoelectric material).

The imaging guide wire of the invention may be used in conjunction with conventional imaging catheters or other types of catheters, such as delivery catheters. Furthermore, the touch sensing catheters of the current invention are suitable for use with any other guide wire available.

The touch sensing catheter allows an operator to obtain tactile information regarding a luminal surface as the catheter is slideably moved along a guide wire to the location of interest. In certain embodiments, the touch sensing catheter is a combination catheter that can perform intraluminal procedures such as delivering implants, ablation, and extraction.

Like the touch sensing guide wire, the touch sensing catheter includes one or more touch sensing elements. As discussed previously, each touch sensing element includes an optical fiber that may comprise a Fiber Bragg Grating. Like the touch sensing guide wire, the sensing elements can be positioned anywhere along and on the inner body of the imaging catheter.

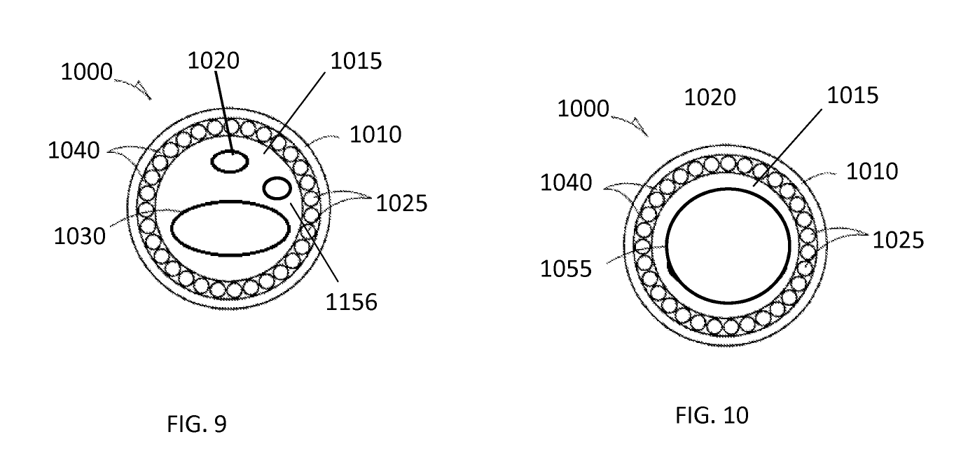

For example, FIG. 9 illustrates a cross-sectional view of an touch sensing catheter 1000 according to one embodiment. The touch sensing catheter 1000 includes sensing elements 1025 that surround an inner body member 1015 of the touch sensing catheter 1000. The sensing elements 1025 are positioned next to each other, parallel to, and along the length of the inner body member 1015. As shown in the cross-sectional view, the sensing elements 1025 are arranged around the circumference of the inner body member 1015 of the touch sensing catheter 1000. The sensing elements 1025 are disposed in binding material 1040. The touch sensing catheter 1000 may be surrounded by an outer catheter sheath or protective coating 1010. The outer catheter sheath or protective coating 1010 can be made from any acoustically transparent resiliently flexible material such as polyethylene or the like, which will permit such transparency while maintaining a sterile barrier around the sensing elements.

Further shown in FIG. 9, the touch sensing catheter 1000 includes a guide wire lumen 1020. The guide wire lumen 1020 receives at least a portion of a guide wire. The touch sensing catheter 1000 can be designed as an over-the-wire catheter or a rapid exchange catheter. Over-the-wire catheters include a guide wire lumen that runs the full length of the catheter. Rapid exchange catheters include a guide wire lumen extending only through a distal portion of the catheter. With respect to the remaining proximal portion of the catheter, the guide wire exits the internal catheter lumen through a guide wire exit port, and the guide wire extends in parallel along the proximal catheter portion.

The touch sensing catheter 1000 may optionally, and as shown in FIG. 9, include one or more tool lumens 1030. The tool lumen 1030 is formed from an inner catheter sheath or member that is disposed within the inner body 1015 of the touch sensing catheter 1000. Through the tool lumen 1030, a catheter tool or device can be introduced into a body lumen, such as blood vessel, for treatment. In addition, the touch sensing catheter may optionally include a removal lumen 1056 that extends from the distal end of the imaging catheter to an opening operably associated with a vacuum source. During intraluminal procedures, a tool element may shave off plaque or other substances from the vessel wall that needs to be removed from the lumen. The shaved-off plaque can be removed from the removal lumen.

FIG. 10 depicts another embodiment of the sensing catheter 1000. In this embodiment, the imaging catheter includes a combined lumen 1055 for receiving the catheter tool or device and the imaging guide wire. The combined lumen 1055 is helpful when the catheter tool or device must also circumscribe the guide wire. For example, implants placed within a body vessel and implant delivery mechanisms are often driven over the guide wire so that the implant may be placed flush against the vessel without the guide wire obstructing implant placement.

Reference will now be made to the specific embodiment of a fiber optic touch sensor for use in conjunction with the aforementioned guide wires and catheters. It is to be understood that this embodiment is not limiting. In certain embodiments, operation of the touch sensor relies on the deformation of a plurality of FBGs held in place by a deformable elastic polymer material. Whenever a force is applied to the sensor, the flexible inner body is deformed and resulted in stretching or compressing the FBGs. By monitoring the wavelength shifts on the plurality of FBGs arranged at certain angles relative to each other in a circle, the magnitude and position of the applied force can be determined.

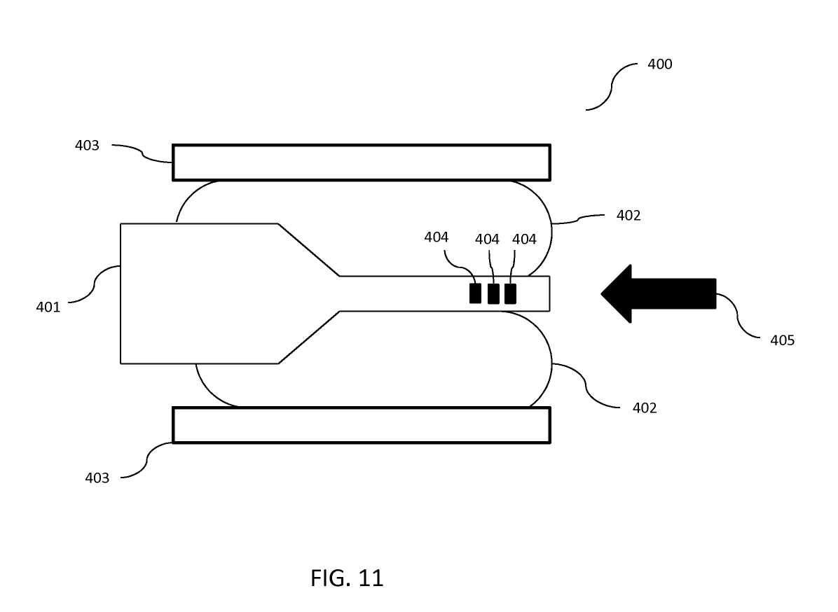

An exemplary touch sensor for use in conjunction with guide wires and catheters of the invention is presented in FIG. 11. An exemplary assembly procedure for preparing such a sensor 400 is now provided. A 30 μm diameter FBG-inscribed optical fiber 401 may be embedded in the center of siliconized rubber 402, an elastomer material, which may then be placed inside a non-magnetized stainless steel tube 403. The FBG inscribed optical fiber is compressed when it comes into contact with an object such as blood vessels, resulting in a reflection wavelength shift of the FBGs 404. The optical fiber was cleaved at a distance of 12-mm from the FBG as shown in FIG. 11. The other end of the optical fiber is a standard single mode fiber with a diameter of 125 μm and is connected to a detection module, such as an FBG interrogator (e.g., Micron Optic Inc., model SM130). Whenever a small force 405 is applied to the sensor 400, the silicone rubber 402 together with the gratings 404 is slightly compressed in the axial direction because the stainless tube 403 restricts the side-way movement of the silicone rubber 402. As a result, the compressive strain induced to the FBG 404 is linearly proportional to the applied force 405. After calibration, the contact force can be determined by monitoring the reflective wavelength of the FBG 404. The Bragg wavelength λ_{B} is given by the equation:

λ_{B}=2n_{eff}Λ,

where n_{eff} and Λ are the effective refractive index and the pitch of the FBG, respectively.

When the grating is under compressive strain, these two parameters are changed. The change of Λ is due to physically shortening in the length of the grating and the change in microfiber's refractive index due to the photoelastic effect. The relationship of the wavelength shift and axial strain can be expressed as:

Δλ/λ_{B}=(1−Pₑ)εₐₓ,

where Pₑ is the constant of the optic-strain coefficient and εₐₓ is the axial strain. The provided touch sensor 400 has a linear response to the compressive strain induced to the grating 404 within the operation range. Ignoring the temperature effect, the shift of the reflected wavelength can be simplified to:

Δλ=−αF,

where F is the applied force in the linear region and a is the sensitivity of the sensor to axial force.

The microfiber may be fabricated by tapering a standard single-mode optical fiber. The fiber can be fed into a heating filament and pulled from one end using a commercial glass processing machine (Vytran's GPX-3400). The fabrication parameters for making a 30 μm fiber taper were as follows: pulling velocity=1-mm/s, input power to filament=29-W, feeding speed at pulling the waist=57.6-μ/s, and taper and waist lengths are all 20-mm. The taper length of the microfiber can be long enough to effectively recouple only the fundamental mode back to the single mode fiber.

A bend-insensitive single-mode fiber (G.657B fiber from Siltec), which contains higher germanium concentration than standard single-mode fiber, may be tapered down from 125 μm to 30 μm. In order to enhance photosensitivity, the taper may be hydrogen loaded at 25° C. for over 60 hours. The 5 mm long FBG can be fabricated using standard phase mask technique with a 1061.5 nm pitch phase mask (Ibsen). A pulsed KrF excimer laser with a wavelength of 248 nm can be used as the writing bream. The pulse energy and pulse rate may be 13 mJ and 200 Hz, respectively. The apodization profile may be realized by scanning the writing beam at the microfiber with a hamming profile at maximum velocity of 5-mm/s over 10 times. The corresponding inverse hamming profile for flattening the DC exposure of the grating can also be used. During this process, the phase mask may be taken out before scanning the writing beam to the grating again.

Silicone rubber (Rhodorsil RTV-573) inside a 0.3-mm thick stainless steel tube with a square cross-section of 2.4 mm×2.4 mm can be used to restrict the deformation of the microfiber in the axial direction for small contact force. Consequently, linear contact force-to-wavelength conversion in the FBG can be obtained. Rhodorsil RTV-573 is a low viscosity liquid silicone that cured at room temperature by adding a curing agent to form a strong flexible silicone rubber. The liquid silicone with a 2% weighted catalyst was first filled into a stainless steel mold. After 24 hours, the silicone rubber will solidify. The FBG was aligned in the center of the tube with the aid of two fiber holders. The optical touch sensor can now be fixed to a guide wire or catheter using the techniques described throughout the disclosure. In the embodiment just described, the touch sensor, upon fixation to the guide wire, would be primarily located at a distal region of the guide wire, in particular, the tip.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are therefore intended to be embraced therein.

1. A device for sensing the surface of a luminal wall, the device comprising:
an elongated body configured to fit within the lumen of a vessel; and
at least one touch sensor located on the elongated body.

2. The device of claim 1, wherein the elongated body is a catheter.

3. The device of claim 1, wherein the elongated body is a guide wire.

4. The device of claim 1, wherein the touch sensor comprises an optical fiber.

5. The device of claim 4, wherein the optical fiber comprises at least one fiber Bragg grating.

6. The device of claim 5, wherein the fiber Bragg grating is an unblazed fiber Bragg grating.

7. The device of claim 5, wherein the fiber Bragg grating is a blazed fiber Bragg grating.

8. The device of claim 1, wherein the touch sensor is located at a distal region of the elongated body.

9. The device of claim 8, wherein the distal region is the distal tip of the elongated body.

10. The device of claim 1, wherein the touch sensor is located at a side region of the elongated body.

11. A method for sensing the surface of a luminal wall, the method comprising,
providing a surface sensing device comprising an elongated body configured to fit within the lumen of a vessel and at least one touch sensor located on the elongated body;
inserting the device into the lumen of a vessel; and
advancing the device until the device contacts a lumen surface.

12. The method of claim 11, wherein the elongated body is a catheter.

13. The method of claim 11, wherein the elongated body is a guide wire.

14. The method of claim 11, wherein the touch sensor comprises an optical fiber.

15. The method of claim 14, wherein the optical fiber comprises at least one fiber Bragg grating.

16. The method of claim 15, wherein the fiber Bragg grating is an unblazed fiber Bragg grating.

17. The method of claim 15, wherein the fiber Bragg grating is a blazed fiber Bragg grating.

18. The method of claim 11, wherein the touch sensor is located at a distal region of the elongated body.

19. The method of claim 18, wherein the distal region is the distal tip of the elongated body.

20. The method of claim 11, wherein the sensor is located at a side region of the elongated body.
